(19) United States
(12) Patent Application Publication
(10) Pub. No.: US 2006/0099707 A1
(43) Pub. Date: May 11, 2006

(54) Automated cell preparation system and method

(76) Inventors: Alan C. Nelson, Gig Harbor, WA (US); Florence W. Patten, Issaquah, WA (US)

Correspondence Address: GEORGE A LEONE, SR, 2150 128TH AVENUE, NW, MINNEAPOLIS, MN, 55448, US

(21) Appl. No.: 10/984,221

(22) Filed: Nov. 9, 2004

(51) Int. Cl. C12N 5/06; C12N 5/02

(52) U.S. Cl. 435/325; 435/289.1

(57) ABSTRACT

An apparatus and method for automated cell preparation is described. A biological cell sample, including large particles and smaller objects of interest, is introduced into a first chamber. The large particles are trapped in the first chamber using a first filter, while smaller objects-of-interest and small particles pass through the first filter into the second chamber where the objects-of-interest are trapped by a second filter having a smaller pore size than the first filter and the small particles pass through the second filter if they are smaller than the objects-of-interest. Debris is purged from the first chamber while the objects-of-interest are trapped in the second chamber. The objects-of-interest are dispensed from the second chamber.

### FIELD OF THE INVENTION

The present invention relates to biological cell preparation in general, and, more particularly, to a system and method for automated cell preparation for cellular objects in liquid suspension.

### BACKGROUND OF THE INVENTION

Specimen preparation for biological cells, for example, in cancer cell analysis using cytology or flow cytometry, has typically consisted of preparing specimens on microscope slides or suspending specimens in a fluid medium. Unfortunately such methods do not promote ease of handling with an acceptable throughput for an automated three-dimensional microscopy system, one example of which is disclosed by Nelson in U.S. Pat. No. 6,522,775 issued Feb. 18, 2003, the contents of which are incorporated by this reference.

### SUMMARY OF THE INVENTION

The present invention provides a system and method for automated cell preparation. A biological cell sample, that may include large particles, smaller objects of interest, and even smaller particles, is introduced into a first chamber. The large particles are trapped in the first chamber using a first filter, while smaller objects-of-interest and the smallest particles pass through the first filter into the second chamber where the objects-of-interest are trapped by a second filter having a smaller pore size than the first filter and the smallest particles pass through the second filter if they are smaller than the objects-of-interest. Debris may be purged from the first chamber while the objects-of-interest are retained in the second chamber. The objects-of-interest may then be dispensed from the second chamber or processed further.

### BRIEF DESCRIPTION OF THE DRAWINGS

FIG. 1 schematically shows an example illustration of a system for automated cell preparation as contemplated by an embodiment of the present invention.

FIG. 2 schematically shows the example illustration of the system and method for automated cell preparation of FIG. 1 in operation as contemplated by an embodiment of the present invention.

FIG. 3 schematically shows an example illustration of a sensor module for use in automated cell preparation as contemplated by an embodiment of the present invention.

FIGS. 4A and 4B schematically show example illustrations of a system for automated cell preparation in operation to clear debris as contemplated by an embodiment of the present invention.

FIG. 5 schematically shows an example illustration of a system for automated cell preparation in operation for staining as contemplated by an embodiment of the present invention.

FIG. 6 schematically shows an example illustration of a system for automated cell preparation in operation to release cells for transfer as contemplated by an embodiment of the present invention.

FIG. 7 schematically shows an example illustration of a system for automated cell preparation in operation to concentrate a cell slurry in preparation for transfer as contemplated by an embodiment of the present invention.

FIG. 8 schematically shows an example illustration of a system for automated cell preparation in operation to optionally reject particles of no interest as contemplated by an embodiment of the present invention.

FIG. 9 schematically shows an example illustration of a capillary receptacle for use in automated cell preparation as contemplated by an embodiment of the present invention.

FIG. 10 schematically shows an example illustration of a capillary receptacle and sensor module for use in automated cell preparation as contemplated by an embodiment of the present invention.

FIG. 11 schematically shows an example illustration of a capillary receptacle placed into a cassette for use in automated cell preparation as contemplated by an embodiment of the present invention.

FIG. 12 schematically shows an example illustration of cassettes in stackable queues for use in automated cell preparation as contemplated by an embodiment of the present invention.

### DETAILED DESCRIPTION OF THE PREFERRED EMBODIMENTS

The invention is described herein with respect to specific examples relating to biological cells, however, it will be understood that these examples are for the purpose of illustrating the principals of the invention, and that the invention is not so limited.

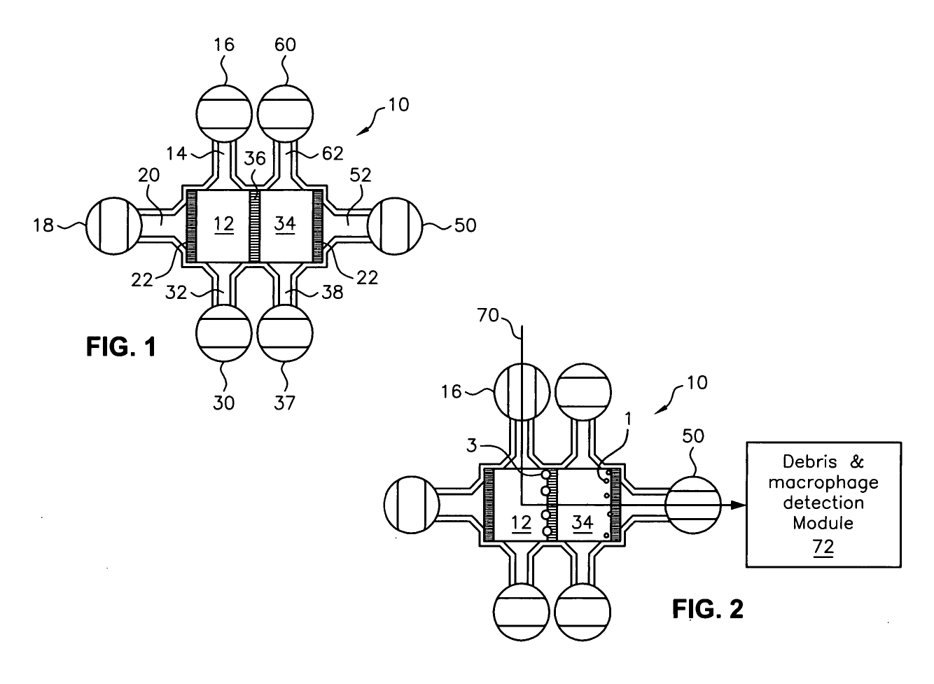

Referring now to FIG. 1, an example of a system for automated cell preparation as contemplated by an embodiment of the present invention is shown. A system for processing a specimen 10 includes a first chamber 12 coupled at a first port 14 to a first valve 16. A second valve 18 is coupled at a second port 20 to the first chamber 12 through a first small pore filter 22. A third valve 30 is coupled at a third port 32 to the first chamber 12. A second chamber 34 is in fluid communication with the first chamber 12, and separated from the first chamber 12 by a large pore filter 36. The second chamber 34 is coupled to a fourth valve 37 at a fourth port 38. A fifth valve 50 is coupled to the second chamber 34 at a fifth port 52 through a second small pore filter 22. A sixth valve 60 is coupled to the second chamber 34 at a sixth port 62, wherein the first through sixth valves operate cooperatively to allow separation of large particles from smaller particles including objects of interest, such as biological cells, when such particles are introduced into the first and second chambers.

The system of the invention is useful in preparing specimens for analyzing various types of biological cells. In one example embodiment, the first small pore filter 22, second small pore filter 22 and large pore filter 36 trap objects of interest including particles, as for example, biological cells, having a diameter in the range of 10 microns to 100 microns. The cell of interest may be selected to be diagnostic of cancer, and/or, the cell of interest may advantageously be a preinvasive cancer cell. The cell of interest may comprise an invasive cancer cell where the cells of interest are utilized in screening a patient for cancer. The cells of interest may also be utilized in determining whether a patient will develop invasive cancer. The invasive cancer cell can be derived from an epithelial cancer. Alternatively, or in addition, the preinvasive cancer cell can be derived from an epithelial cancer. The epithelial cancer may be advantageously selected from the group consisting of lung cancer, throat cancer, cervical cancer, ovarian cancer, breast cancer, prostate cancer, skin cancer, cancer of the gastrointestinal tract, lymphatic cancer and bone cancer.

Referring now to FIG. 2, the system for automated cell preparation of FIG. 1 is shown in operation as contemplated by an embodiment of the present invention. As indicated by directional arrow 70 a biological cell sample, including large particles 3 and smaller objects of interest 1, is introduced into the first chamber 12 by opening the first valve 16. When the fifth valve 50 is opened in cooperation with the second valve 16, the larger particles 3 are trapped by the large pore filter 36, while smaller objects of interest 1, such as biological cells, pass through the large pore filter. Fluid from the second chamber 34 passes through the fifth valve 50 to a detection module 72 including a debris and macrophage detection system.

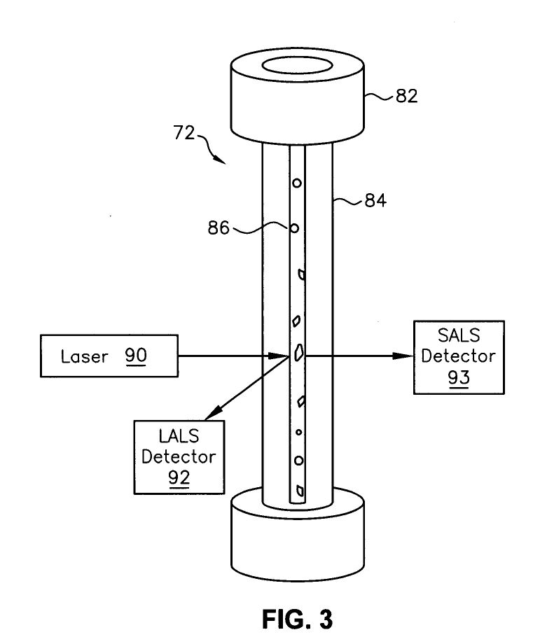

Referring now to FIG. 3, an example illustration of a sensor module for use in automated cell preparation as contemplated by an embodiment of the present invention is schematically shown. A sensor module 80 advantageously includes a set of automatic exchange interconnects 82 attached at opposing ends of a capillary tube 84. A plurality of particles 86 flows through the capillary tube 84. A laser diode 90 is positioned for illuminating particles in the capillary tube 84 so as to produce small angle light scattering (SALS) for particle size detection and large angle light scattering (LALS) for particle nuclear complexity detection. A plurality of silicone photodiode detectors 92, 93 may advantageously be positioned to receive scattered light including small angle light scattering for particle size detection and large angle light scattering for particle nuclear complexity detection. In one useful embodiment, the capillary tube 84 comprises circular or rectangular fused silica capillary tubing.

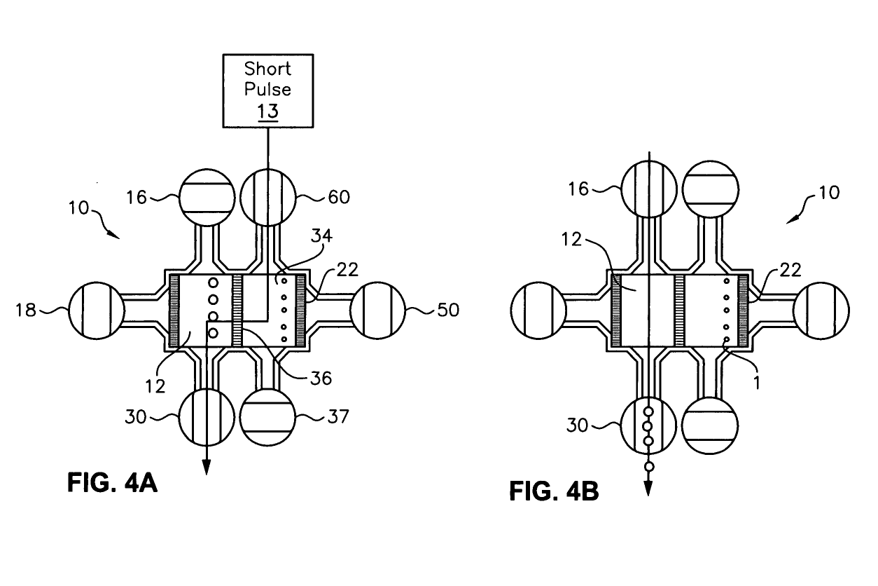

Referring now to FIG. 4A an example illustration of a system for automated cell preparation in operation to clear debris as contemplated by an embodiment of the present invention is schematically shown. The processing system 10 is configured to have the third valve 30 and sixth valve 60 both opened while using a pulse of fluid 13 to free debris from the large pore filter 36 100 μm filter while cells of interest 1 remain near the 10 μm right small pore filter 22. In one example, the large pore filter 36 may be a 100 μm filter while the right small pore filter 22 may have pores of about 10 μm. One useful clearing fluid includes a reagent of 50% EtOH.

Referring now to FIG. 4B, an example illustration of a system for automated cell preparation in operation to clear debris as contemplated by an embodiment of the present invention is schematically shown. The processing system 10 is configured to have the first valve 16 and third valve 30 both opened to clear debris from the first chamber 12 using clearing fluid. As above, one useful clearing fluid includes a reagent of 50% EtOH.

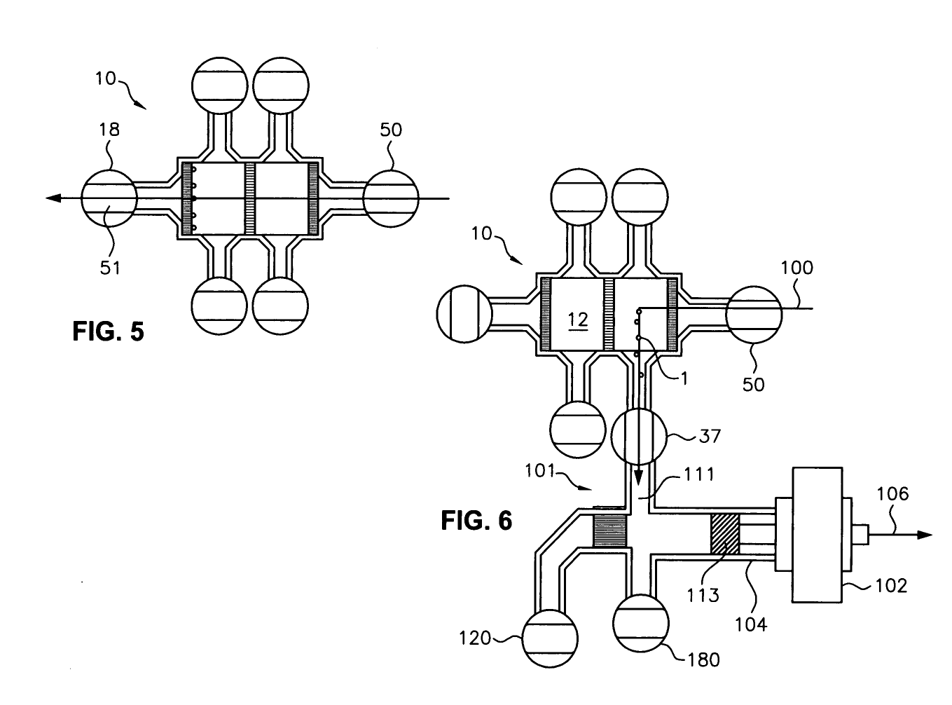

Referring now to FIG. 5 an example illustration of a system for automated cell preparation in operation for staining as contemplated by an embodiment of the present invention is schematically shown. As an example of one possible staining process, staining proceeds along the following steps:

At step Stain 1-1, the sample is pre-stained twice, and rinsed once with a reagent comprising 50% EtOH, where the flow 51 is in a first direction.

At step Stain 1-2 the sample is pre-stained twice, and rinsed twice with a reagent comprising double distilled (DD) H₂O, where the flow is in a second direction opposite the first direction.

At step Stain 1-3, the sample is pre-stained once, and rinsed 3 times with a reagent comprising DDH₂O, where the flow is in the first direction.

At step Stain 1-4, a timed stain of 1 minute is carried out with a reagent/stain comprising Hematoxylin.

Step Stain 1-5 is a single post-stain and a single rinse with a reagent comprising DDH₂O, where the flow is in the first direction.

Step Stain 1-6 is a single post-stain and double rinse with a reagent comprising DDH₂O+4% (by volume) ammonia, where the flow is in the second direction.

Step Stain 1-7 is a single post-stain and a triple rinse with a reagent comprising DDH₂O, where the flow is in the first direction.

This completes the first rinse and first stain procedure. Additional protocols for counterstains, antibody based probes, and so on can be added and implemented analogous to steps Stain 1-4 thru Stain 1-7 with appropriate reagents and steps adapted as required

Still referring to FIG. 5, the staining procedure may be followed by a solvent exchange procedure including the steps of:

At step Solvent exchange-1 solvent is exchanged with solvent comprising 50% ethanol (EtOH). Cells are then allowed to equilibrate by transmembrane diffusion.

At step Solvent exchange-2 solvent is exchanged with solvent comprising 80% EtOH. Cells are then allowed to equilibrate by transmembrane diffusion.

At step Solvent exchange-3 solvent is exchanged with solvent comprising 100% EtOH. Cells are then allowed to equilibrate by transmembrane diffusion.

At step Solvent exchange-4 solvent is again exchanged with solvent comprising 100% EtOH. Cells are then allowed to equilibrate by transmembrane diffusion. The second rinse is a factor of safety for full cellular dehydration, and for competing the EtOH exchange.

At step Solvent exchange-5 solvent is exchanged with solvent comprising 50% EtOH and 50% xylene. Cells are then allowed to equilibrate by transmembrane diffusion.

At step Solvent exchange-6 solvent is again exchanged with solvent comprising 50% EtOH and 50% xylene to insure transition. Cells are then allowed to equilibrate by transmembrane diffusion.

At step Solvent exchange-7 solvent is exchanged with solvent comprising 100% xylene. Cells are then allowed to equilibrate by transmembrane diffusion.

At step Solvent exchange-8 solvent is exchanged with solvent comprising 100% xylene. Cells are then allowed to equilibrate by transmembrane diffusion.

At step Solvent exchange-9 solvent is exchanged for a third rinse/exchange with solvent comprising 100% xylene. Cells are then allowed to equilibrate by transmembrane diffusion.

At step Solvent exchange-10, prior to releasing cells for transfer, solvent is exchanged with a solvent comprising 100% xylene while pulsing in the second direction, and completing solvent exchange to xylene.

Referring now to FIG. 6, an example illustration of a system for automated cell preparation in operation to release cells for transfer as contemplated by an embodiment of the present invention is schematically shown. A cell concentration module 101 includes a syringe pump 102 and a passage 111 coupled to the output of the fourth valve 37 through an input leg 104. A lower valve 180 is closed. Dehydrated cells 1 are transferred to the cell concentration module including the syringe pump 102 as indicated by directional arrow 100. The syringe pump 102 is aspirated (plunger 113 is withdrawn) as indicated by directional arrow 106 while providing positive bias pressure on input leg.

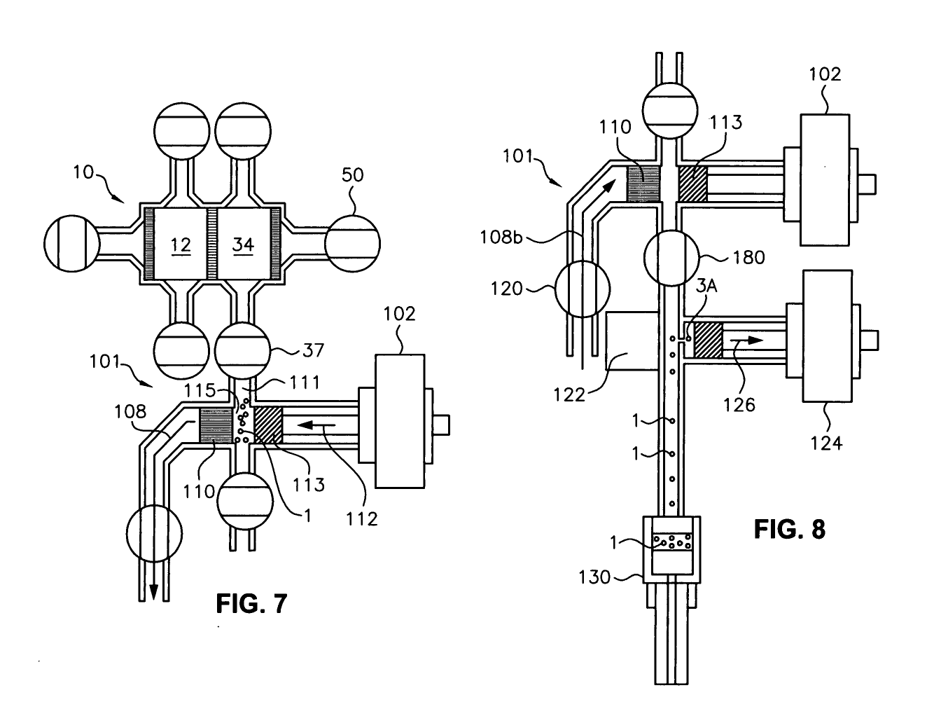

Referring now to FIG. 7, an example illustration of a system for automated cell preparation in operation to concentrate cell slurry in preparation for transfer as contemplated by an embodiment of the present invention is schematically shown. In this configuration the fourth valve 37 is closed after the cells 1 have been transferred to passage 111. A shunt 108 is connected through a filter 110 to passage 111 containing the cells 1. By dispensing cell pre-concentration module syringe pump 102 by moving the plunger forward as indicated by directional arrow 112, the xylene is shunted to waste along with smaller particles of no interest and a concentrated cell suspension 115 is created on the filter 110 surface. In operation, the pre-concentration syringe pump 102 plunger 113 is pushed against the filter 110.

Referring now to FIG. 8, an example illustration of a system for automated cell preparation in operation to selectively remove cells of no interest while sending cells of interest to a sample transport device as contemplated by an embodiment of the present invention is schematically shown. The contents of the cell concentration module 101 are dispensed using a backflush 108b through the filter 110 to free cells from the filter surface. The lower valve 180 is opened. The flushing continues until cells arrive at the capillary receptacle 130. Cells may be allowed to flow past the optional detection module 122 and, when passing cells of no interest 3A are detected they are aspirated by the shunt pump 124 as indicated by directional arrow 126 for later disposal. Before processing another sample, all upstream fluidics receive a precision cleaning protocol. All filters receive multiple backwashes and all lines are fully deproteinated in accordance with standard practices.

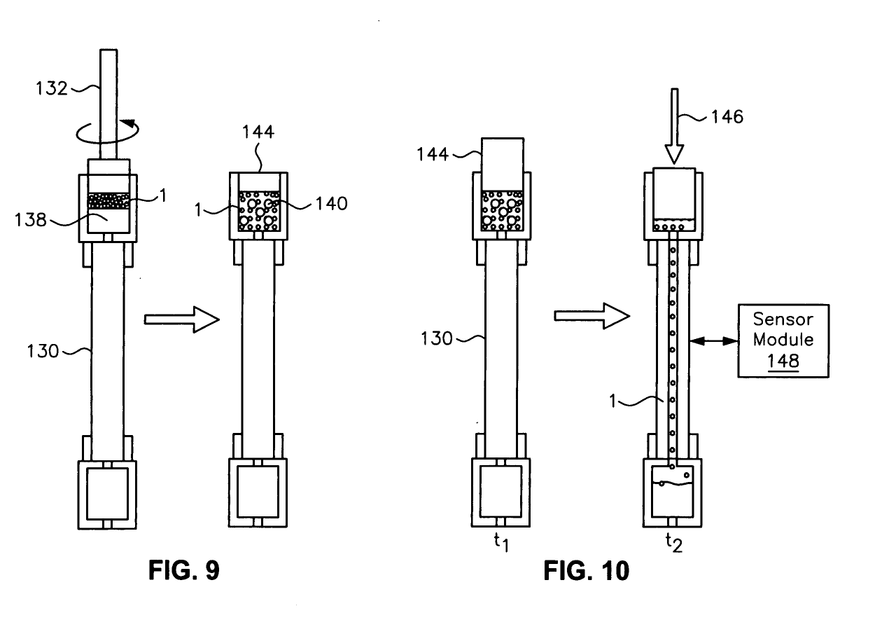

Referring now to FIG. 9, an example illustration of a capillary receptacle for use in automated cell preparation as contemplated by an embodiment of the present invention is schematically shown. Xylene is removed by evaporation with vacuum to create a cell layer for subsequent blending. In a vacuum, the cells 1 are blended in the capillary receptacle 130 with an optical gel 138, creating many vacuoles and pockets 140. A mixer 132 may preferably comprise a single use molded plastic component that is discarded when operation is complete.

Referring now to FIG. 10, an example illustration of a capillary receptacle and sensor module for use in automated cell sample preparation as contemplated by an embodiment of the present invention is schematically shown. At time t₁, while maintained in a vacuum, a disposable piston/cap 144 is placed in the capillary receptacle now containing cells embedded in optical gel. At time t₂, force 146 is applied and sensor module 148 is used to verify proper mean load spacing of the cells 1 in optical gel. Sensor module 148 may comprise an automated visioning system, microscope or equivalents.

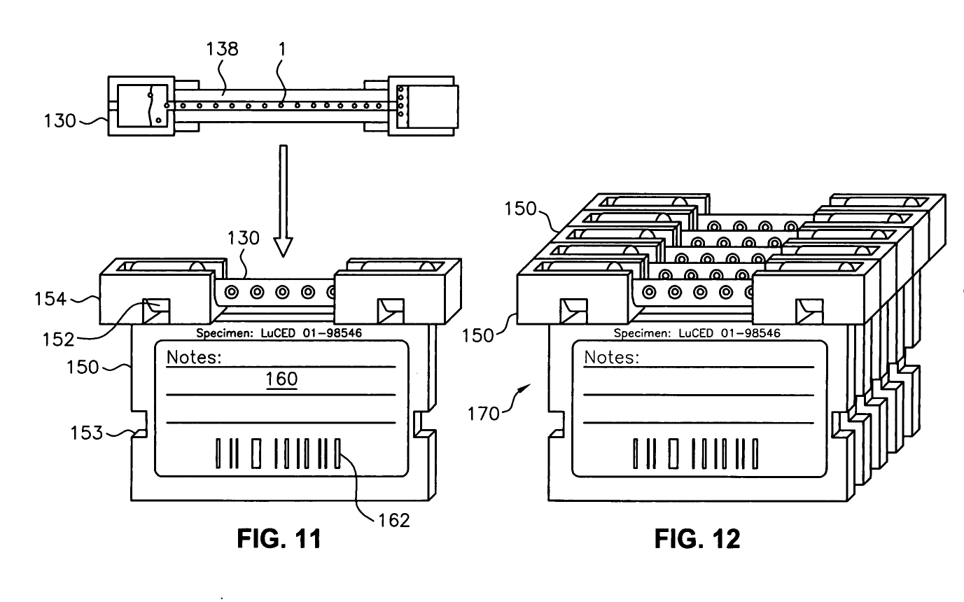

Referring now to FIG. 11, an example illustration of a capillary receptacle placed into a cassette for use in automated cell preparation as contemplated by an embodiment of the present invention is schematically shown. The capillary receptacle 130 is placed in a protective handling cassette 150. The protective handling cassette 150 includes access points 152 for robotic extraction of the capillary receptacle 130, registry points 153 for automatic alignment verification and grip points 154 for cassette manipulation. The protective handling cassette 150 may also advantageously include identifying indicia 160 including, for example, a bar code 162. The protective handling cassette 150 has a generally rectangular shape so as to allow stacking with one or more additional handling cassettes.

Referring now to FIG. 12, an example illustration of cassettes in stackable queues for use in automated cell analysis as contemplated by an embodiment of the present invention is schematically shown. Cassettes 150 are placed in stackable queues 170 that buffer the input of the (not shown) 3D microscopy reader. Cassette queues are also removable for reading.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, and devices and reconstruction algorithms, and that various modifications, both as to the equipment details and operating procedures, may be accomplished without departing from the true spirit and scope of the present invention.

What is claimed is:

1. A method for automated cell preparation comprising the steps of:
introducing a biological cell sample including large particles and smaller objects of interest into a first chamber;
trapping the large particles in the first chamber using a first filter, while smaller objects of interest pass through the first filter into a second chamber and are trapped by a second filter having a smaller pore size than the first filter, wherein the second chamber is in fluid communication with the first chamber, and separated from the first chamber by the first filter;
freeing debris from the first filter while the smaller objects of interest are trapped in the second chamber;
then dispensing the smaller objects of interest from the second chamber into a concentration module,
concentrating the smaller objects of interest to form a cell concentrate;
flushing the cell concentrate to transport the smaller objects of interest to a capillary receptacle; and
blending the remaining portion of the smaller objects of interest in the capillary receptacle with an optical gel to allow viewing of the smaller objects of interest in a microscope.

2. The method of claim 1 wherein the step of freeing debris further comprises using a pulse of clearing fluid to free debris from the first filter.

3. The method of claim 2 wherein the clearing fluid comprises ethanol (EtOH).

4. A method for automated cell preparation comprising the steps of:
introducing a biological cell sample including large particles and smaller objects of interest into a first chamber;
trapping the large particles in the first chamber using a first filter, while smaller objects of interest pass through the first filter into a second chamber and are trapped by a second filter having a smaller pore size than the first filter, wherein the second chamber is in fluid communication with the first chamber, and separated from the first chamber by the first filter; and
passing fluid from the second chamber through the second filter to a detection module including a debris and macrophage detection system, wherein the debris and macrophage detection system includes a capillary tube for receiving fluid, a laser light source positioned for illuminating particles in the capillary tube so as to produce small angle light scattering (SALS) for particle size detection and large angle light scattering (LALS) for particle nuclear complexity detection, and a plurality of photodetectors positioned to receive scattered light including small angle light scattering for particle size detection and large angle light scattering for particle nuclear complexity detection.

5-6. (canceled)

7-11. (canceled)

11. The method of claim 1 further comprising the step of capping and mounting the capillary receptacle in a cassette.

12. A system for processing a specimen comprising:
a first chamber coupled at a first port to a first valve;
a second valve coupled at a second port to the first chamber through a first small pore filter;
a third valve coupled at a third port to the first chamber;
a second chamber in fluid communication with the first chamber, and separated from the first chamber by a large pore filter, the second chamber coupled to a fourth valve at a fourth port;
a fifth valve coupled to the second chamber at a fifth port through a second small pore filter; and
a sixth valve coupled to the second chamber at a sixth port, wherein the first through sixth valves operate cooperatively to allow separation of large particles from smaller particles including objects of interest.

13. The system of claim 12 wherein the first large pore filter and second small pore filter trap particles of interest having a diameter in the range of 100 microns to 10 microns.

14. The system of claim 12 wherein the objects of interest comprise cells.

15. The system of claim 12 wherein the fifth valve operates to pass fluid from the second chamber to a detection system.

16. The system of claim 15 wherein the detection system comprises a debris and macrophage detection system.

17. The system of claim 15 wherein the detection system comprises a flow cytometer including a plurality of flow cells in a capillary tube, a laser diode positioned for illuminating particles in the capillary tube so as to produce small angle light scattering for particle size detection and large angle light scattering for particle nuclear complexity detection and having a plurality of photodiode detectors positioned to receive scattered light including small angle light scattering for particle size detection and large angle light scattering for particle nuclear complexity detection.

18. The system of claim 17 wherein the capillary tube comprises circular or rectangular fused silica capillary tubing having a polyimide coating.

19. The system of claim 12 wherein the fourth valve operatively couples the second chamber to a syringe pump.

20. The system of claim 19 wherein the syringe pump is connected to a filtered shunt where the syringe pump operates to create a concentrated cell suspension by pumping waste through the filtered shunt.

21. The system of claim 20 wherein the syringe pump is also in fluid communication with a particle flow tube, where the particle flow tube operates to dispense the contents of cell concentration system.

22. The system of claim 21 wherein the particle flow tube uses fluid flow to pass objects by a detection system and, on detection of events of no interest, objects of no interest are aspirated into a shunt pump.

23. The system of claim 22 wherein the particle flow tube couples at a dispensing end to a capillary receptacle.

24. The system of claim 23 wherein the capillary receptacle comprises a capillary tube having an exchange interconnect at a first end.

25. A protective handling cassette comprising:
a cassette housing having with a capillary gripper;
a pair of opposing clips on the top for releasably holding a capillary receptacle;
a plurality of access points for robotic extraction of the capillary;
a plurality of registration points for automatic alignment verification; and
a plurality of grip points for cassette manipulation.

26. The protective handling cassette of claim 25 wherein the cassette has a generally rectangular shape so as to allow stacking with one or more additional handling cassettes.

27. The method of claim 1 wherein the smaller objects of interest comprise at least one cell having a diameter in the range of 10 microns to 100 microns.

28. The method of claim 27 wherein the at least one preinvasive cancer cell is derived from an epithelial cancer.

29. The method of claim 28 wherein the epithelial cancer is selected from the group consisting of lung cancer, throat cancer, cervical cancer, ovarian cancer, breast cancer, prostate cancer, skin cancer, cancer of the gastrointestinal tract, lymphatic cancer and bone cancer.

30. (canceled)

31. The method of claim wherein the at least one preinvasive cancer cell is selected from the group consisting of lung and throat cancer, cervical cancer, breast cancer, cancer of the gastrointestinal tract, lymphatic cancer and bone cancer.

32. The method of claim 1 wherein the objects of interest comprise at least one invasive cancer cell.

33. The method of claim 32 wherein the at least one invasive cancer cell is derived from an epithelial cancer.

34. The method of claim 33 wherein the epithelial cancer is selected from the group consisting of lung cancer, throat cancer, cervical cancer, ovarian cancer, breast cancer, prostate cancer, skin cancer, cancer of the gastrointestinal tract, lymphatic cancer and bone cancer.

35. The method of claim 32 wherein the at least one invasive cancer cell is derived from a neuroendocrine cancer.

36. The method of claim 35 wherein the neuroendocrine cancer is selected from the group consisting of lung and throat cancer, cervical cancer, breast cancer, cancer of the gastrointestinal tract, lymphatic cancer and bone cancer.
